(19) United States
(12) Patent Application Publication
(10) Pub. No.: US 2023/0126239 A1
(43) Pub. Date: Apr. 27, 2023

(54) NORRIN REGULATION OF PLASMALEMMA VESICLE-ASSOCIATED PROTEIN AND USE TO TREAT MACULAR DEGENERATION

(71) Applicant: Retinal Solution, LLC, Rochester, MI (US)

(72) Inventors: Kimberly Drenser, Rochester, MI (US); Michael T. Trese, Rochester, MI (US); Antonio Capone, Rochester, MI (US)

(73) Assignee: Retinal Solutions, LLC, Rochester, MI (US)

(21) Appl. No.: 17/910,467

(22) PCT Filed: Mar. 8, 2021
(86) PCT No.: PCT/US2021/021281

§ 371 Date: Sep. 9, 2022

Related U.S. Application Data
(60) Provisional application No. 62/986,871, filed on Mar. 9, 2020.

(51) Int. Cl. A61K 38/18; A61K 49/00; A61P 27/02

(52) U.S. Cl. CPC A61K 38/18; A61K 49/0043; A61K 49/006; A61P 27/02

(57) ABSTRACT

A method is provided to limit inter-cellular leakage between cells in retinal or choroidal vasculature. As a result, an ocular disorder in which ocular or choroidal edema occurs based on leakage of the Adherens Junctions or Tight Junctions is readily treated. The method is particularly well-suited for usage in response to the blood-retinal barrier (BRB) compromise. A method is also provided for the reduction of plasmalemma vesicle-associated protein (PLVAP), which causes transcytosis and pinicytotic leakage. In a particular application, fluid collection under retinal pigment epithelial cells in wet macular degeneration is reduced; a condition currently without effective clinical treatments.

### CROSS REFERENCE TO RELATED APPLICATIONS

This application claims priority benefit of U.S. Provisional Application Ser. No. 62/986,871 filed 9 Mar. 2020, the contents of which are hereby incorporated by reference.

### FIELD OF THE INVENTION

The present invention is directed generally to methods of regulating plasmalemma vesicle-associated protein (PLVAP) and endothelial diaphragms in retinal tissue by applying to such tissue norrin protein; and in particular, to inhibit PLVAP in retinal vasculature to limit macular degeneration associated with compromised retinal vasculature Tight Junctions.

### BACKGROUND OF THE INVENTION

Various retinopathies cause regions of leakage induced edema within the retina. To compensate for the lack of oxygen & nutrients in the ischemic tissue, cytokines involved in permeability are excessively up-regulated. The over-expression of vascular endothelial growth factor (VEGF) causes an increase in vessel permeability due to induced expression of plasmalemma vesicle-associated protein (PLVAP), leading to exacerbation of cellular permeability. Over-expression of VEGF also causes an increase in vessel permeability due to VEGF-induced translocation of Tight Junction transmembrane protein Claudin 5 to cytosol which breaks down retinal vasculature Tight Junctions.

The increased permeability of vasculature in the eye can result in retinal diseases such as macular edema, wet age-related macular degeneration, retinal vein occlusion, diabetic retinopathy, radiation retinopathy, and Familial Exudative Vitreoretinopathy (FEVR). While clinical characterization of the causes of such retinal diseases such as diabetes have been studied, little attention has been paid to addressing the weakened inter-cellular junctions in retinal vasculature. The edema associated with vasculature leakage can cause complications such as macular edema and exudative retinal detachment.

The epithelium including retinal pigment epithelium function to separate blood in the circulatory system from other tissues. The epithelium are sites of exchange as well as barriers, for the transit of ions and molecules between tissues and the circulatory system of the organism. Complexes between adjacent cells include Tight Junctions and Adherens junctions. Vertebrate epithelial cells exhibit Tight Junctions that lie apical to Adherens Junctions. Tight junctions have an organizing role in epithelial polarization and establish an apico-lateral barrier to the diffusion of solutes through the intracellular space (gate function). Tight junctions also restrict the movement of lipids and membrane proteins between the apical and the basolateral membrane (fence function). Tight Junctions are highly ordered membrane contact sites, comprising a network of intra-membrane fibrils. Tight Junctions include transmembrane proteins, including occludin, claudin-5, and junctional adhesion molecules (JAMs), and a number of cytoplasmic peripheral proteins. These are shown schematically in prior art FIG. 1. While the transmembrane proteins mediate cell-cell adhesion, the cytosolic tight junction plaque contains various types of proteins (e.g., PDZ proteins, such as the ZO (Zona Occludens) family) that link tight junction transmembrane proteins to the underlying cytoskeleton. These adapters also recruit regulatory proteins, such as protein kinases, phosphatases, small GTPases and transcription factors, to the tight junctions. As a result, structural (Actin and Spectrin) and regulatory (Actin-binding proteins, GTPases and kinases) proteins are juxtaposed with transmembrane proteins. This protein scaffolding facilitates the assembly of highly ordered structures, such as junctional complexes or signaling patches that regulate epithelial cell polarity, proliferation and differentiation. This scaffolding is also operative in retinal pigment epithelium.

Plasmalemma vesicle-associated protein (PLVAP) is the only protein that forms endothelial diaphragms used in transcellular transport to exchange substances between the blood plasma and interstitial fluid. PLVAP is a type II integral membrane glycoprotein with a molecular weight of 50,000; it has a short (27aa) intracellular N-terminal domain, a single-span transmembrane domain, and a long (380-aa) C-terminal extracellular domain. The homodimers, i.e., diaphragm fibrils, form sieve-like structures by creating a central knob. The diaphragms work as physical filters in transcellular transport. Damage to the microvascular endothelial cells leads to the breakdown of the blood-retinal barrier (BRB), which increases microvascular permeability, resulting in the extravasation of blood to the macula. PLVAP is the only protein that forms endothelial diaphragms. PLVAP is very low in the normal blood-retinal barrier, however, vascular endothelial growth factor (VEGF) induces PLVAP expression, leading to the exacerbation of cellular permeability.

VEGF inhibitors are commonly used to treat leaky blood vessels, however, there are several concerns associated with their use, especially in the case of chronic suppression of VEGF. DRCR Protocol S is a way of measurement based on PDR treated with PRP laser or monthly anti-VEGF; at 5 years the peripheral field of vision was not statistically significantly different between the two groups. There is also the “ischemic index” by Justice Ehlers which shows persistent capillary drop out in eyes treated with anti-VEGF (Sun et al., Ophthalmology, 2019; 126 (1): 87-95)

Norrin is a ligand for the Frizzled receptor subtype 4 (Fz4). Norrin binds Fz4 with nanomolar affinity (Xu, et al, Cell, 2004; 116:883-895; Clevers, Curr Biol, 2004; 14:R436-437; Nichrs, Dev Cell, 2004; 6:453-454). Norrin interaction with Fz4 is dependent on the cell surface receptor LRP5. (Xu, 2004). Frizzled receptors are coupled to the β-catenin canonical signaling pathway. The inactivation of glycogen synthase kinase (GSK) 3β and Axin through frizzled receptor binding stabilizes β-catenin, which subsequently accumulates in the cell nucleus and activates the transduction of target genes that are crucial in the G1-S-phase transition, such as cyclin D1 or c-Myc. (Willert et al., Curr Opin Genet Der, 1998: 8:95-102). Suppression of norrin activity has been shown to preclude angiogenesis associated with ocular disease (US 2010/0129375). Norrin protein has not been implicated in the treatment of wet age-related macular degeneration associated with leaking vasculature in the eye.

Thus, there exists a need for a method to treat macular degeneration associated with vasculature leakage. There further exists a need for a method to treat clinical disorders associated with retinal edema as seen in vasoprolifertive diseases such as macular edema, wet age-related macular degeneration, retinal vein occlusion, diabetic retinopathy, and radiation retinopathy. There also exists a need to treat fluid pockets in macular degeneration, as well as FEVR. The present invention is directed to these, as well as other, important needs in the art.

### SUMMARY OF THE INVENTION

A method is provided for tightening inter-cellular junctions in a retinal or choroidal vessel cells. The method includes exposing the retinal or choroidal vessel cells to norrin, and allowing sufficient time for the norrin to selectively decrease expression of plasmalemma vesicle-associated protein (PLVAP) or vascular endothelial growth factor (VEGF) in the retinal or choroidal vessel cells to tighten the inter-cellular junctions.

A method is provided for tightening inter-cellular junctions in a retinal or choroidal vessel cells. The method includes exposing the retinal or choroidal vessel cells to norrin, and allowing sufficient time for the norrin to translocate Claudin 5 to cell membranes in the retinal or choroidal vessel cells to tighten the inter-cellular junctions.

### BRIEF DESCRIPTION OF THE DRAWINGS

These and other aspects of the present invention will be elucidated in the accompanying drawings and following detailed description of the invention.

FIGS. 1A and 1B are prior art schematics of a cell having intact cell junctions (FIG. 1A) and weakened or disrupted cell junctions (FIG. 1B), showing the function of VEGF in changing the pathway of certain highlighted cellular pathways;

FIG. 2A is a schematic of a microvascular cell with substantial permeability given the significant expression of PLVAP;

FIG. 2B is a schematic of a possible pathway for the effectiveness of the present invention to reduce cellular expression of PLVAP;

FIG. 3 is a graph showing PLVAP expression in VEGF and Norrin treated human retinal microvascular endothelial cells (HRMECs);

FIG. 4 is a graph showing Norrin inhibition of VEGFA induced PLVAP gene expression; and

FIGS. 5A and 5B are retinal micrographs depicting Evans Blue leakage in eyes of an oxygen induced retinopathy (OIR) model of a mouse, with an uninjected eye (FIG. 5A) and an eye injected with norrin (FIG. 5B), the images taken 4 days after norrin injection, where bright (white) is indicative of vascular leakage and the injected eye shows preservation of the capillary network.

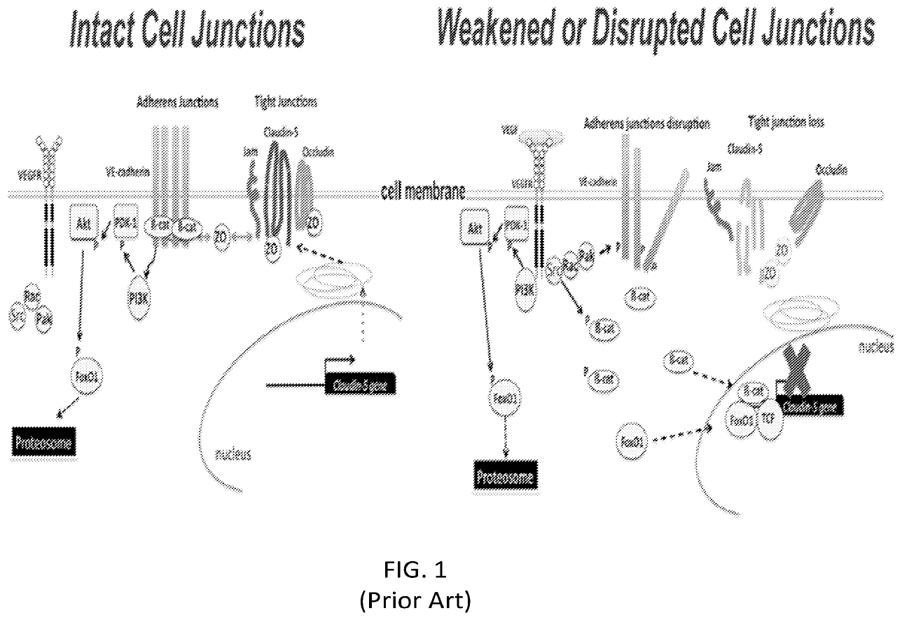

### DETAILED DESCRIPTION OF THE INVENTION

The present invention has utility as a method to limit inter-cellular leakage between cells in retinal or choroidal vasculature. As a result, an ocular disorder in which ocular or choroidal edema occurs based on leakage of the Adherens Junctions or Tight Junctions is readily treated. The present invention is particularly well-suited for usage in response to the blood-retinal barrier (BRB) compromise. A method is also provided for the reduction of plasmalemma vesicle-associated protein (PLVAP), which causes transcytosis and pinicytotic leakage. In a particular application, fluid collection under retinal pigment epithelial cells in wet macular degeneration is reduced; a condition currently without effective clinical treatments. The invention will be described in detail below. Those skilled in the art will appreciate that the description given herein is for exemplary purposes only and is not intended in any way to limit the scope of the invention.

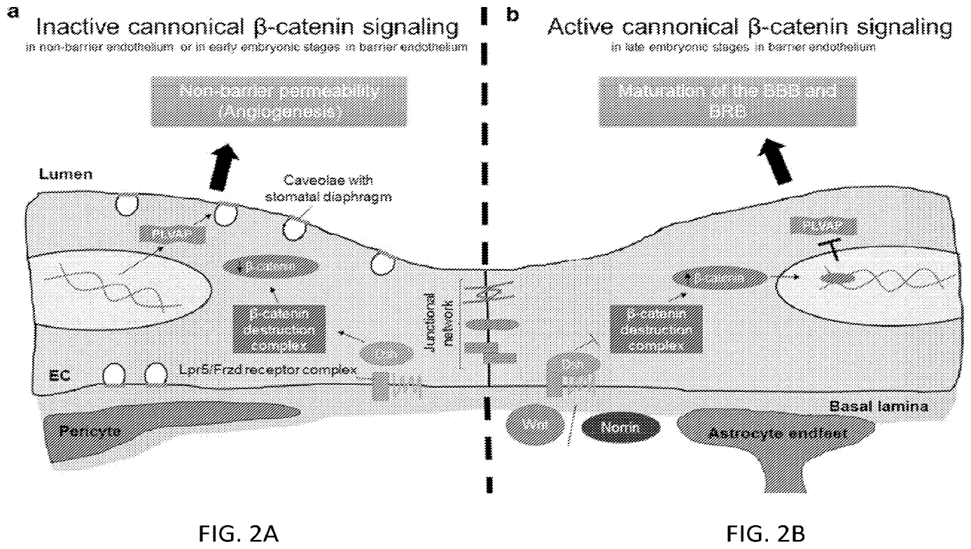

Without intending to be bound to a particular theory of operation, the binding of norrin protein to a Lpr5/Frzd (frizzled-4) receptor of a retinal epithelial cell limits degradation of beta-catetin, that then accumulates and localizes to the epithelial cell nucleus and subsequently inhibits the expression of PLVAP proteins that form endothelial diaphragms in the retinal epithelial cell. By limiting the expression of PLVAP proteins and the formation of endothelial diaphragms, cellular permeability across the blood brain barrier or blood retinal barrier is significantly reduced. This is shown schematically in FIG. 2B, which is contrasted with the schematic of FIG. 2A in which a receptor of a retinal epithelial cell is not treated by norrin and therefore expresses the PLVAP protein and has a high degree of cellular permeability.

The following definitions are used herein with respect to the understanding of the present invention.

“Administering” is defined herein as a means of providing norrin protein or a composition containing norrin to a subject retina. Such an administration can be by any route including, without limitation, oral, transdermal (e.g., oral mucosa), by injection (e.g., subcutaneous, intravenous, parenterally, intraperitoneally, intraocular), by inhalation (e.g., oral or nasal), or topical (e.g., eyedrops, cream, etc.). Pharmaceutical preparations are, of course, given by forms suitable for each administration route.

By “alteration” is meant a change (increase or decrease) in the expression levels or activity of a gene or polypeptide as detected by standard art known methods such as those described herein. As used herein, an alteration includes at least a 10% change in expression levels, preferably a 25% change, more preferably a 40% change, and most preferably a 50% or greater change in expression levels.

By “analog” is meant a molecule that is not identical, but has analogous functional or structural features to norrin protein. For example, a polypeptide analog retains the biological activity of a corresponding naturally-occurring norrin, while having certain biochemical modifications that enhance the analog's function relative to a naturally occurring polypeptide. Such biochemical modifications could increase the analog's protease resistance, solubility, membrane permeability, or half-life, without altering, for example, ligand binding. An analog may include an unnatural amino acid.

In this disclosure, “comprises,” “comprising.” “containing” and “having” and the like can have the meaning ascribed to them in U.S. patent law and can mean “includes,” “including,” and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “control” is meant a standard or reference status.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “fragment” is meant a portion of norrin. This portion contains, preferably, at least 40%, 50%, 60%, 70%, 80%, or 90% of the entire length of the 133 amino acid residues of the native human norrin polypeptide. A fragment may contain 40, 50, 60, 70, 80, 90, 100,110, 120, 130 or even the complete 133 amino acids.

By “truncate” is meant to include a fragment of norrin that has a polypeptide terminus cleavage of the norrin protein of up 40 amino acid residues.

By an “isolated polypeptide” is meant a polypeptide analog of norrin that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

Norrin is meant to define a polypeptide or fragment thereof having at least about 85% amino acid identity to NCBI Accession No. NP_000257.1, as shown below, and having the ability to bind the frizzled-4 receptor of retinal epithelial cells.

gil4557789lreflNP_000257.11 norrin precursor
[Homo sapiens]
(SEQ ID NO. 1)
MRKHVLAASFSMLSLLVIMGDTDSKTDSSFIMDSDPRRCMRHHYVDSISH
PLYKCSSKMVLLARCEGHCSQASRSEPLVSFSTVLKQPFRSSCHCCRPQT
SKLKALRLRCSGGMRLTATYRYILSCHCEECNS

As used herein, “obtaining” as in “obtaining an agent” includes synthesizing, purchasing, or otherwise acquiring the agent.

The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e″³ and e″¹⁰⁰ indicating a closely related sequence.

As used herein, the terms “treat,” “treated.” “treating.” “treatment,” and the like refer to reducing or ameliorating a disorder and/or symptoms associated therewith BRB compromise.

Typically a therapeutically effective dosage should produce a serum concentration of compound of from about 0.1 ng/ml to about 50-100 μg/ml.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Norrin is a 133 amino acid long protein that is secreted into the extracellular space. Two primary domains define the general norrin protein structure: a signal peptide directs localization of the molecule; and a cysteine-knot motif provides the tertiary confirmation required for frizzled-4 receptor binding. (Meitinger, T, et al, Nat Genet, 1993; 5:376-380; Berger, W, et al. Hum Mol Genet, 1996; 5:51-59). Truncates and fragments of norrin that retain the ability to bind frizzled-4 receptor are operative herein. In some inventive embodiments a truncate or fragment of norrin retains the cysteine-knot motif.

The importance of the cysteine knot-motif is highlighted by computer modeling that demonstrates the requirement of disulfide bonds between the cysteine residues in forming the structural confirmation of norrin. However, mutations in regions other than the cysteine knot-motif produce incomplete protein folding and result in familial exudative vitreoretinopathy (FEVR) and related vitreoretinopathies.

In certain inventive embodiments a −24 residue N-terminus truncate of norrin is present, with the following amino acid sequence:

(SEQ ID NO. 2)
KTDSSFIMDSDPRRCMRHHYVDSISHPLYKCSSKMVLLARCEGHCSQAS
RSEPLVSFSTVLKQPFRSSCHCCRPQTSKLKALRLRCSGGMRLTATYRY
ILSCHCEECNS (Accession # Q00604)

It has been found that some fragments and truncations such as SEQ ID NO: 2 have improved solubility compared to norrin.

The invention further embraces variants and equivalents which are substantially homologous to norrin and still retain the ability to selectively bind the frizzled-4 receptor. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid, or one neutral amino acid by another neutral amino acid.

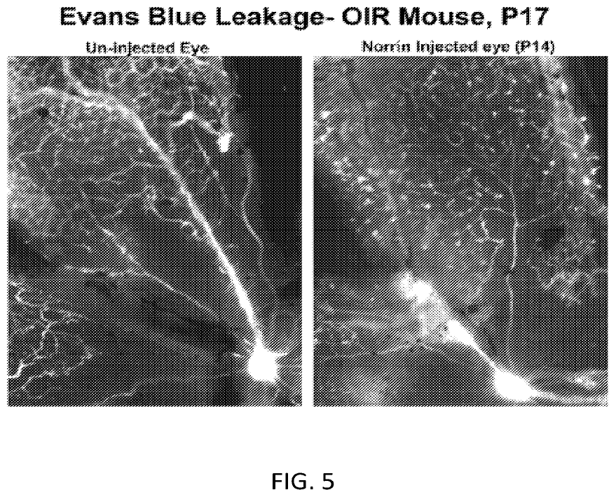

The norrin of the present invention can be recombinant norrin, natural norrin, or synthetic norrin retaining frizzled-4 binding properties. It will be recognized in the art that some amino acid sequences of the invention can be varied without significant effect of the structure or function of the protein. Thus, the invention further includes variations of the norrin which show substantial activity; such mutants include deletions, insertions, inversions, repeats, and type substitutions. Norrin mutants operable herein illustratively include amino acid substitutions relative to SEQ ID NO: 1 of R64E (SEQ ID NO. 3). Optionally the biologically active peptide is a multiple mutant relative to SEQ ID NO: 1: T27A (SEQ ID NO. 4), S28A (SEQ ID NO. 5), S29A (SEQ ID NO. 6); P36A (SEQ ID NO. 7), R37A (SEQ ID NO. 8), R38A (SEQ ID NO. 9); Y120A (SEQ ID NO. 10), R121A (SEQ ID NO. 11), Y122A (SEQ ID NO. 12); or H127A (SEQ ID NO. 13), E129A (SEQ ID NO. 14), E130A (SEQ ID NO. 15); or combinations thereof. Any amino acid mutated in a multiple mutation is operable as a single mutation. Other sequence mutations operative herein are illustrated in FIGS. 5 and 6 of Smallwood, P M, et al., J Biol Chem, 2007: 282:4057-4068 or Ke, J et al. Genes& Dev. 2013: 27: 2305-2319. These mutations include K86E: MRKHVLAASFSMLSLLVIMGDTDSKTDSSFIMDSDPRRCMRHHYVDSISHPLYKCSS KMVLLARCEGHCSQASRSEPLVSFSTVLEQPFRSSCHCCRPQTSKLKALRLRCSGGM RLTATYRYILSCHCEECNS (SEQ ID NO. 16), R90E: MRKHVLAASFSMLSLLVIMGDTDSKTDSSFIMDSDPRRCMRHHYVDSISHPLYKCSS KMVLLARCEGHCSQASRSEPLVSFSTVLKQPFESSCHCCRPQTSKLKALRLRCSGGM RLTATYRYILSCHCEECNS (SEQ ID NO. 17), R97E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCEPQTSKLKALRLRCSGGM RLTATYRYILSCHCEECNS (SEQ ID NO. 18), K102E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSELKALRLRCSGGM RLTATYRYILSCHCEECNS (SEQ ID NO. 19), K104E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLEALRLRCSGGM RLTATYRYILSCHCEECNS (SEQ ID NO. 20), and R115E: MRKHVLAASFSMLSLLVIMGDTDSKTDSSFIMDSDPRRCMRHHYVDSISHPLYKCSS KMVLLARCEGHCSQASRSEPLVSFSTVLKQPFRSSCHCCRPQTSKLKALRLRCSGGM ELTATYRYILSCHCEECNS (SEQ ID NO. 21). It is appreciated that other mutations at different amino acid sites are similarly operable. It is further appreciated that mutation of the conserved amino acid at any particular site is preferably mutatated to glycine or alanine. It is further appreciated that mutation to any neutrally charged, charged, hydrophobic, hydrophilic, synthetic, non-natural, non-human, or other amino acid is similarly operable.

Modifications and changes are optionally made in the structure (primary, secondary, or tertiary) of the Norrin protein which are encompassed within the inventive compound that may or may not result in a molecule having similar characteristics to the exemplary polypeptides disclosed herein. It is appreciated that changes in conserved amino acid bases are most likely to impact the activity of the resultant protein. However, it is further appreciated that changes in amino acids operable for receptor interaction, resistance or promotion of protein degradation, intracellular or extracellular trafficking, secretion, protein-protein interaction, post-translational modification such as glycosylation, phosphorylation, sulfation, and the like, may result in increased or decreased activity of an inventive compound while retaining some ability to alter or maintain a physiological activity. Certain amino acid substitutions for other amino acids in a sequence are known to occur without appreciable loss of activity.

In making such changes, the hydropathic index of amino acids are considered. According to the present invention, certain amino acids can be substituted for other amino acids having a similar hydropathic index and still result in a polypeptide with similar biological activity. Each amino acid is assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Without intending to be limited to a particular theory, it is believed that the relative hydropathic character of the amino acid determines the secondary structure of the resultant polypeptide, which in turn defines the interaction of the polypeptide with other molecules. It is known in the art that an amino acid can be substituted by another amino acid having a similar hydropathic index and still obtain a functionally equivalent polypeptide. In such changes, the substitution of amino acids whose hydropathic indices are within .+−.2 is preferred, those within .+−.1 are particularly preferred, and those within .+−.0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Lu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu).

The norrin and analogs can be further modified to contain additional chemical moieties not normally part of the protein. Those derivatized moieties can improve the solubility, the biological half-life, absorption of the protein, or binding affinity. The moieties can also reduce or eliminate any undesirable side effects of the proteins and the like. An overview for those moieties can be found in Remington's Pharmaceutical Sciences, 20th ed., Mack Publishing Co., Easton, Pa. (2000).

The isolated norrin described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. (Zoeller et al., Proc. Nat'l. Acad. Sci. USA 81:5662-5066 (1984) and U.S. Pat. No. 4,588,585).

According to the present invention, the Tight Junctions and Adherens Junctions of retinal epithelial cells with compromised junctions are exposed to a dosage of norrin, a truncate or fragment thereof. After norrin exposure the resulting cells have demonstrably lower levels of PLVAP expression and VEGF, specifically VEGFA165b, induced PLVAP expression. The present invention thus reverses the effects of PLVAP and VEGF on retinal and choroidal epithelial cells. Retinal edema, retinal detachment, macular edema, macular degeneration, wet age-related macular degeneration, retinal vein occlusion, diabetic retinopathy, radiation retinopathy, and FEVR associated with retinal or choroidal epithelial cellular permeability is thereby reduced.

Over-expression of VEGF causes an increase in vessel permeability due to VEGF-induced translocation of Tight Junction membrane protein Claudin 5 to cytosol which breaks down retinal vasculature Tight Junctions. According to embodiments of the present invention, the Tight Junctions of retinal epithelial cells with compromised junctions are exposed to a dosage of norrin, a truncate or fragment thereof. After norrin exposure the resulting cells exhibit reduced or no vascular leakage, including capillaries with an intact BRB. Norrin translocates Claudin 5 from cytosol to the cell membrane, reestablishing Tight Junctions, thereby counteracting the effects of VEGF illustratively including translocation of Claudin 5 to cytosol. Retinal edema, retinal detachment, macular edema, macular degeneration, wet age-related macular degeneration, retinal vein occlusion, diabetic retinopathy, radiation retinopathy, and FEVR associated with retinal or choroidal epithelial cellular permeability is thereby reduced.

Norrin truncate of SEQ ID NO: 2 is observed to be effective in decreasing endothelial diaphragm forming PLVAP proteins at concentrations of 10 to 1000 ng/ml.

The present invention is also directed to pharmaceutical compositions comprising an effective amount of norrin alone or in combination with a pharmaceutically acceptable carrier, excipient or additive. Particularly favored derivatives are those that increase the bioavailability of norrin administered to a mammal (e.g., by allowing ocularly of choroidal administered norrin to be more readily absorbed into the blood) or which enhance delivery of the norrin to a biological compartment (e.g., the retina) relative to the native protein.

To prepare the pharmaceutical compositions according to the present invention, a therapeutically effective amount of norrin is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others.

Norrin is also administered with an adjunct therapeutic such as an anti-VEGF agent. An anti-VEGF agent operative herein illustratively includes bevacizumab ranibizumab small molecules that inhibit the tyrosine kinases stimulated by VEGF such as lapatinib, sunitinib, sorafenib, axitinib, pazopanib, or a combination thereof. A combination therapeutic provided that includes an anti-VEGF agent and a norrin. It has been surprisingly found that by simultaneously suppressing VEGF binding to a cell and stimulation of Tight Junction and Adherens Junction protein expression that the efficacy of conventional anti-VEGF agents is enhanced. By way of example, anti-VEGF agents are typically effective in approximately 75% of subjects with an indication of macular edema secondary to diabetes. This effectiveness is increased by more to more than 85% with simultaneous administration of a norrin.

Solutions or suspensions used for ocular, parenteral, intradermal, subcutaneous, or topical application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Administration in the form of a liquid oral preparation uses a carrier in a form such as suspensions, elixirs and solutions, suitable carriers and additives including water, glycols, oils, alcohols, flavoring agents, preservatives, coloring agents and the like may be used. For solid oral administration, preparations are provided in a form such as powders, tablets, capsules, and for solid preparations such as suppositories, suitable carriers and additives including starches, sugar carriers, such as dextrose, mannitol, lactose and related carriers, diluents, granulating agents, lubricants, binders, or disintegrating agents. If desired, the tablets or capsules may be enteric-coated or sustained release by standard techniques. Norrin is provided in a solid dose is lyophilized form or in pelletized solution droplets.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, poly anhydrides, polyglycolic acid, collagen, polyorthoesters, and polylactic acid. Methods for preparation of such formulations will be apparent to those skilled in the art.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

Formulations suitable for topical administration to the skin may be presented as ointments, creams, gels and pastes including the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier will usually comprise sterile water or aqueous sodium chloride solution, though other ingredients including those which aid dispersion may be included. Of course, where sterile water is to be used and maintained as sterile, the norrin and carriers must also be sterilized. Injectable suspensions may also be prepared, in which case appropriate liquid carriers, suspending agents and the like may be employed.

Formulations suitable for parenteral or ocular administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include topical, ocular, parenteral, intramuscular, intravenous, sub-cutaneous, intrachoroidal, or transdermal (which may include a penetration enhancement agent).

Application of the subject therapeutics may be local, so as to be administered at the site of interest. Various techniques can be used for providing the subject norrin at the site of interest, such as injection, use of catheters, trocars, projectiles, pluronic gel, stents, sustained drug release polymers, or other device which provides for internal access. Where an organ or tissue is accessible because of removal from the patient, such organ or tissue may be bathed in a medium containing the subject norrin, the subject norrin may be painted onto the organ, or may be applied in any convenient way.

Norrin may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment. More specifically, the norrin is administered through an ocular device suitable for direct implantation into the vitreous of the eye. Such devices of the present invention are surprisingly found to provide sustained controlled release of various norrin to treat the eye without risk of detrimental local and systemic side effects. An object of the present ocular method of delivery is to maximize the amount of drug contained in an intraocular device while minimizing its size in order to prolong the duration of the implant. See, e.g., U.S. Pat. Nos. 5,378,475; 5,773,019; 6,001,386; 6,217,895, 6,375,972, and 6,756,058.

Other methods of delivery of norrin include: an ocular delivery system that could be applied to an intra-ocular lens to prevent inflammation or posterior capsular opacification, an ocular delivery system that could be inserted directly into the vitreous, under the retina, or onto the sclera, and wherein inserting can be achieved by injecting the system or surgically implanting the system, a sustained release drug delivery system, and a method for providing controlled and sustained administration of an agent effective in obtaining a desired local or systemic physiological or pharmacological effect comprising surgically implanting a sustained release drug delivery system at a desired location.

Examples include, but are not limited to the following: a sustained release drug delivery system comprising an inner reservoir containing norrin, an inner tube impermeable to the passage of the agent, the inner tube having first and second ends and covering at least a portion of the inner reservoir, the inner tube sized and formed of a material so that the inner tube is capable of supporting its own weight, an impermeable member positioned at the inner tube first end, the impermeable member preventing passage of the agent out of the reservoir through the inner tube first end, and a permeable member positioned at the inner tube second end, the permeable member allowing diffusion of the agent out of the reservoir through the inner tube second end. A method for administering norrin to a segment of an eye, includes implanting a sustained release device to deliver norrin to the vitreous of the eye or choroid, or an implantable, sustained release device for administering a compound of the invention to a segment of an eye or choroid; a sustained release drug delivery device includes a) a drug core containing norrin; b) at least one unitary cup essentially impermeable to the passage of the agent that surrounds and defines an internal compartment to accept the drug core, the unitary cup including an open top end with at least one recessed groove around at least some portion of the open top end of the unitary cup; c) a permeable plug which is permeable to the passage of norrin, the permeable plug is positioned at the open top end of the unitary cup wherein the groove interacts with the permeable plug holding it in position and closing the open top end, the permeable plug allowing passage of the agent out of the drug core, through the permeable plug, and out the open top end of the unitary cup. A sustained release norrin delivery device includes an inner core norrin having a desired solubility and a polymer coating layer, the polymer layer being permeable to norrin, wherein the polymer coating layer completely covers the inner core.

Norrin may be administered as microspheres. For example, norrin may be purchased from R&D Systems, Minneapolis, Minn., or cloned, expressed and purified is loaded into biodegradable microspheres substantially as described by Jiang, C, et al, Mol. Vis., 2007; 13:1783-92 using the spontaneous emulsification technique of Fu, K et al, J. Pharm. Sci., 2003: 92:1582-91. Microspheres are synthesized and loaded by dissolving 200 mg of 50:50 poly(lactide-co-glycolic acid) (PLGA) in 5 ml of 4:1 volume ratio trifluoroethanol:dichloromethane supplemented with 8 mg magnesium hydroxide to minimize protein aggregation during encapsulation. 10 μg norrin may be reconstituted in 300 μl 7 mg bovine serum albumin (BSA) and 100 mg docusate sodium (Sigma-Aldrich, St. Louis, Mo.) dissolved in 3 ml PBS. The solution may be vortexed and poured into 200 ml of 1% (w/v) polyvinyl alcohol (PVA, 88% hydrolyzed) with gentle stirring. Microspheres may be hardened by stirring for three hours, collected by centrifugation, and washed three times to remove residual PVA. If the microspheres are not to be immediately injected they are rapidly frozen in liquid nitrogen, lyophilized for 72 h, and stored in a dessicator at −20° C. Norrin containing microspheres exhibit average diameters of 8 μιη as determined by a particle size. Norrin may also be administered by intravitreal injection. For example, norrin in solution, may be packaged into microspheres as described above, or expressed in cells, or in purified form in solution may be exposed to the retina by intravitreal injection substantially as described by Jiang, 2007. Intravitreal injection may be performed under general anesthesia using an ophthalmic operating microscope (Moller-Wedel GmbH, Wedel, Germany) using beveled glass micro-needles with an outer diameter of approximately 100 μm. Microsphere suspensions are prepared in PBS at 2 and 10% (w/v) and briefly vortexed immediately before injection to ensure a uniform dispersion. A 30-gauge hypodermic needle may be used to perforate the sclera 1.5 mm behind the limbus. Five microliters of test sample is optionally injected by way of this passage into the vitreous using a 50 μl Hamilton Syringe (Hamilton Co, Reno, Nev.). To ensure adequate delivery and prevent shock the needle is held in place for one min after the injection is completed and subsequently withdrawn slowly. In addition, paracentesis may be simultaneously performed to relieve pressure and thereby prevent reflux.

Norrin may also be administered by delivery to the retina by a controlled release delivery system. An implantable controlled release delivery system is described in U.S. Patent Application Publication 2005/0281861 and is packaged into such as system at 100 μg per final formulated capsule. For example, a norrin containing drug delivery systems may be placed in the eye using forceps or a trocar after making a 2-3 mm incision in the sclera. Alternatively, no incision may be made and the system placed in an eye by inserting a trocar or other delivery device directly through the eye. The removal of the device after the placement of the system in the eye can result in a self-sealing opening. One example of a device that is used to insert the implants into an eye is disclosed in U.S. Patent Application Publication No. 2004/0054374 which is incorporated herein by reference. The location of the system may influence the concentration gradients of therapeutic component or drug surrounding the element, and thus influence the release rates (e.g., an element placed closer to the edge of the vitreous may result in a slower release rate). Thus, it is preferred if the system is placed near the retinal surface or in the posterior portion of the vitreous.

Preferred unit dosage formulations are those containing a daily dose or unit, daily sub-dose, as hereinabove recited, or an appropriate fraction thereof, of the administered ingredient.

The dosage regimen for norrin invention is based on a variety of factors, including the degree of BRB leakage, the route of administration, ocular volume, macular separation volume, and the particular norrin employed. Thus, the dosage regimen may vary widely, but can be determined routinely using standard methods.

In certain embodiments, norrin is administered once daily; in other embodiments, norrin is administered twice daily; in yet other embodiments, norrin is administered once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

Pharmaceutically acceptable carriers, excipients, or diluents illustratively include saline, buffered saline, dextrose, water, glycerol, ethanol, sterile isotonic aqueous buffer, and combinations thereof.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as PLGA, polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

Examples of preservatives include, but are not limited to, parabens, such as methyl or propyl p-hydroxybenzoate and benzalkonium chloride.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Any of the above-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

When norrin is administered as a pharmaceutical to humans or animals, norrin can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, norrin is administered in an amount sufficient to reduce or eliminate symptoms associated with retinal edema or macular degeneration or FEVR.

Exemplary ocular dose ranges include 0.00001 mg to 250 mg per day, 0.0001 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In certain inventive embodiments, the therapeutically effective dosage produces an ocular concentration of norrin of from about 0.1 ng/ml to about 50-100 μg/ml. In certain inventive embodiments, 50 nM to 1 μM of an agent is administered to a subject eye. In related embodiments, about 50-100 nM, 50-250 nM, 100-500 nM, 250-500 nM, 250-750 nM, 500-750 nM, 500 nM to 1 μM, or 750 nM to 1 μM of an norrin is administered to a subject eye.

Determination of an effective amount is well within the capability of those skilled in the art, especially in light of the detailed disclosure provided herein. Generally, an efficacious or effective amount of a norrin is determined by first administering a low dose of the agent(s) and then incrementally increasing the administered dose or dosages until a desired effect (e.g., reduce or eliminate symptoms associated with retinal edema, macular degeneration, macular edema, wet age-related macular degeneration, retinal vein occlusion, diabetic retinopathy, radiation retinopathy, and FEVR) is observed in the treated subject, with minimal or acceptable toxic side effects. Applicable methods for determining an appropriate dose and dosing schedule for administration of a pharmaceutical composition of the present invention are described, for example, in Goodman and Oilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

The following are in vitro examples of how norrin can significantly decrease PLVAP expression to inhibit the endothelial cell depletion and microvascular permeability which are for purposes of illustration and are not intended to limit the scope of the present invention.

### In Vitro Assays.

### Example 1: PLVAP Expression in VEGF and Norrin Treated Cells

Human retinal microvascular endothelial cells (HRMECs) were cultured in DMEM supplemented with 10% (v/v) heat-inactivated FBS (Nichirei Biosciences Inc., Tokyo, Japan), 100 U/mL penicillin, 100 μg/mL streptomycin (Life Technologies Gibco, France. The cell cultures were incubated on collagen-coated tissue culture plates Transwell® (Corning, N.Y., N.Y.) in a humidified atmosphere of 5% CO₂ at 37° C.

HRMECs were cultured for 14 to 21 d on a Lab-Tek chamber plate (Corning). H₂O₂ (500 μmol/L) was administered to the basolateral side of the Transwell®. To some plates nothing was added (Control), or VEGF (100 ng/mL) or norrin truncate (SEQ ID NO: 2) (250 ng/ml) or both VEGF and norrin (100 ng/ml and 250 ng/ml, respectively) was added to the apical medium 30 min prior to H₂O₂ treatment. After 24 h of treatment with the VEGFA+/− Norrin, the cells were washed twice with cold PBS and fixed with cold acetone (Wako Pure Chemical Industries, Osaka, Japan) for 10 min. The cells were then removed from the Transwell® and mounted on slides.

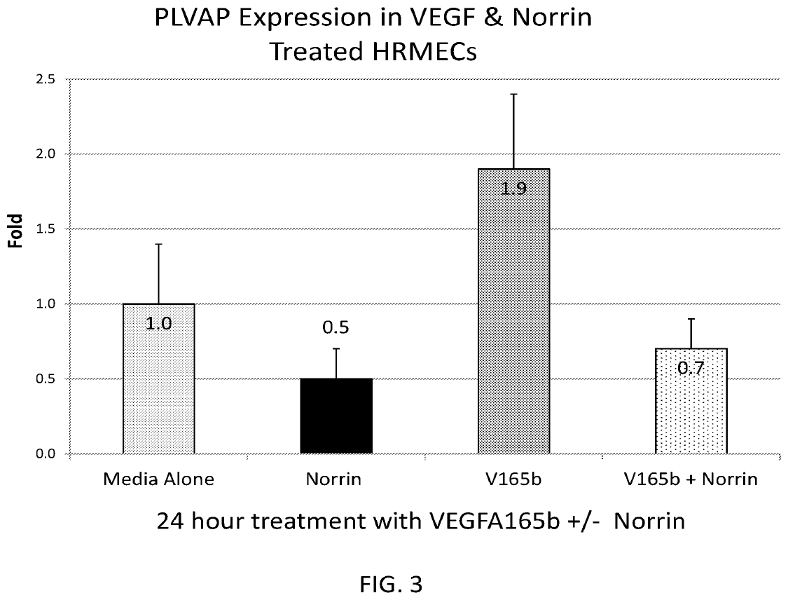

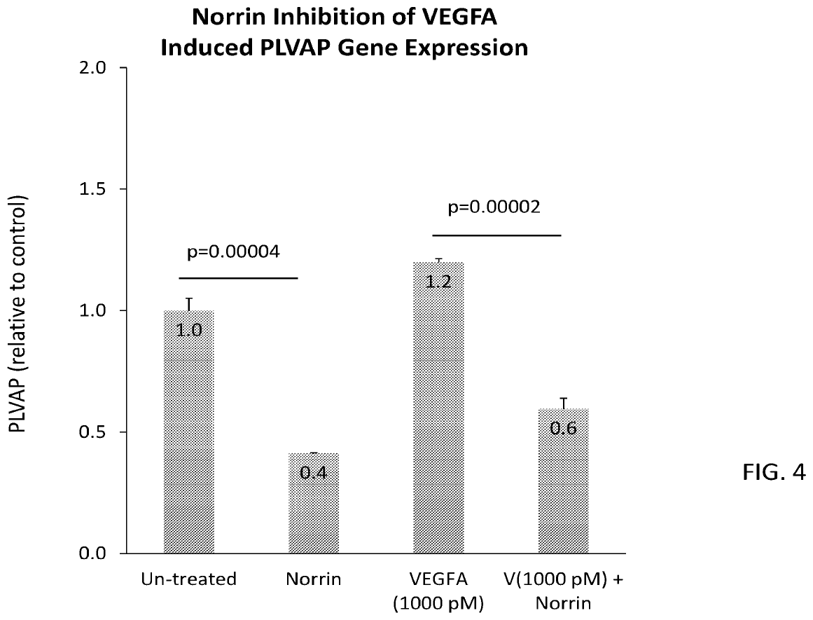

The amount of PLVAP expressed in the treated cells is tested. As shown in the graphs of FIG. 3 and FIG. 4, compared to the control, Norrin significantly decreases the expression of PLVAP. In contrast, VEGF, specifically, VEGFA165b significantly increases the PLVAP expression. Surprisingly, norrin significantly decreases VEGFA165b induced PLVAP expression.

### Example 2: In Vivo Assays of Vessel Leakage in OIR MICE

The mouse Oxygen Induced Retinopathy Model (OIR) is used to create ischemic retina so the changes in vascular morphology and function can be assessed. Raising mice in a high oxygen environment creates areas of avascular retina. Once returned to normal oxygen environment, vessels become leaky and grow in an unregulated fashion. The amount of leakage can be visualized by systemically injecting a fluorescent dye (Evans Blue or fluoroscein) and then viewing the retina under a microscope. In OIR mice, Evans blue dye can be seen leaking from retinal vessels with high levels of PLVAP (left panel of FIG. 5). However, in OIR eyes injected with norrin after the OIR induction of avascular retina, Evans blue dye is confined to vessels (right panel of FIG. 5). Images were taken 4 days after norrin injection in the right eye.

As a person skilled in the art will recognize from the previous detailed description and from the figures and claims, modifications and changes can be made to the preferred embodiments of the invention without departing from the scope of this invention defined in the following claims.

1. A method of tightening inter-cellular junctions in a retinal or choroidal vessel cells, the method comprising:
exposing the retinal or choroidal vessel cells to a norrin mutant having at least 85% amino acid identity to SEQ ID NO. 1 and retaining a cysteine-knot motif of the native norrin protein and capable of binding to the retinal or choroidal vessel cells; and
allowing sufficient time for said norrin mutant to selectively decrease expression of plasmalemma vesicle-associated protein (PLVAP) or vascular endothelial growth factor (VEGF) in the retinal or choroidal vessel cells to tighten the inter-cellular junctions.

2. The method of claim 1, further comprising diagnosing macular degeneration associated with fluid leakage from a retinal vessel defined by retinal vessel cells prior to the exposing step.

3. The method of claim 1, further comprising diagnosing at least one of retinal vein occlusion, diabetic retinopathy, radiation retinopathy, FEVR, or combinations thereof associated with fluid leakage from a retinal vessel defined by retinal vessel cells prior to the exposing step.

4. The method of claim 1, wherein said norrin mutant is a polypeptide of SEQ ID. NO. 2.

5. The method of claim 1, wherein said norrin mutant is a fragment of SEQ ID. NO. 1 that binds a frizzled-4 receptor of the retinal vessel cells.

6. The method of claim 1, wherein said norrin mutant is recombinant.

7. The method of claim 1, wherein the retinal vessel cells are in vivo in a subject.

8. The method of claim 7, wherein exposing step is by intraocular injection, systemic administration, or topical administration.

9. (canceled)

10. (canceled)

11. The method of claim 7, wherein said subject is human.

12. The method of claim 7, wherein said subject is one of: cow, horse, sheep, pig, goat, chicken, cat, dog, mouse, guinea pig, hamster, rabbit, rat, or a cell derived from one of the aforementioned.

13. The method of claim 1, further comprising bringing a dye into contact with the retinal or choroidal vessel cells after the allowing sufficient time for said norrin to decrease expression of PLVAP or VEGF to qualify a tightness of the inter-cellular junctions.

14. The method of claim 13, wherein said dye is an immunostain for PLVAP or VEGF, Evans Blue dye, or fluroscein.

15. (canceled)

16. A method of tightening inter-cellular junctions in a retinal or choroidal vessel cells comprising:
exposing the retinal or choroidal vessel cells to a norrin mutant having at least 85% amino acid identity to SEQ ID NO. 1 and retaining a cysteine-knot motif of the native norrin protein and capable of binding to the retinal or choroidal vessel cells; and
allowing sufficient time for said norrin to translocate Claudin 5 to cell membranes in the retinal or choroidal vessel cells to tighten the inter-cellular junctions.

17. The method of claim 16, further comprising diagnosing at least one of retinal vein occlusion, diabetic retinopathy, radiation retinopathy, FEVR, or combinations thereof associated with fluid leakage from a retinal vessel defined by retinal vessel cells prior to the exposing step.

18. The method of claim 16, further comprising diagnosing macular degeneration associated with fluid leakage from a retinal vessel defined by retinal vessel cells prior to the exposing step.

19. The method of claim 16, further comprising bringing a dye into contact with the retinal or choroidal vessel cells after the allowing sufficient time for said norrin to translocate Claudin 5 to cell membranes in the retinal or choroidal vessel cells to qualify a tightness of the inter-cellular junctions.

20. The method of claim 16, wherein said exposing step is by intraocular injection, systemic administration, or topical administration.

21. (canceled)

22. (canceled)

23. (canceled)

24. The method of claim 16, wherein said norrin mutant is a polypeptide of SEQ ID. NO. 2.

25. The method of claim 16, wherein said norrin mutant is a fragment of SEQ ID. NO. 1 that binds a frizzled-4 receptor of the retinal vessel cells.

26. The method of claim 16, wherein said norrin mutant is recombinant.


SEQUENCE LISTING (21 sequences)

SEQ ID NO: 1 - protein, 133 aa, Homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 2 - protein, 109 aa, Artificial sequence
  Synthetic
KTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 3 - protein, 133 aa, Artificial sequence
  Synthetic
MRKHVLAASFSMLSLLVIMGDTDSKTDSSFIMDSDPRRCMRHHYVDSISHPLYKCSSKMVLLAECEGHCSQASRSEPLVSFSTVLKQPFRSSCHCCRPQTSKLKALRLRCSGGMRLTATYRYILSCHCEECNS

SEQ ID NO: 4 - protein, 133 aa, Artificial sequence
  Synthethic
MRKHVLAASFSMLSLLVIMGDTDSKADSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 5 - protein, 133 aa, Artificial sequence
  Synthetic
MRKHVLAASFSMLSLLVIMGDTDSKTDASFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 6 - protein, 133 aa, Artificial sequence
  Synthetic
MRKHVLAASFSMLSLLVIMGDTDSKTDSAFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 7 - protein, 133 aa, Artificial sequence
  Synthetic
MRKHVLAASFSMLSLLVIMGDTDSKTDSSFIMDSDARRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 8 - protein, 133 aa, Artificial sequence
  Synthetic
MRKHVLAASFSMLSLLVIMGDTDSKTDSSFIMDSDPARCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 9 - protein, 133 aa, Artificial sequence
  Synthetic
MRKHVLAASFSMLSLLVIMGDTDSKTDSSFIMDSDPRACMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 10 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATARYILSCHCEECNS

SEQ ID NO: 11 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYAYILSCHCEECNS

SEQ ID NO: 12 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRAILSCHCEECNS

SEQ ID NO: 13 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCACEECNS

SEQ ID NO: 14 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCAECNS

SEQ ID NO: 15 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEACNS

SEQ ID NO: 16 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLEQPFRSSCHCCRPQTSKLKALRLRCSGGMRLTATYRYILSCHCEECNS

SEQ ID NO: 17 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFESSCHCCRPQTSKLKALRLRCSGGMRLTATYRYILSCHCEECNS

SEQ ID NO: 18 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCEPQTSKLKALRLRCSGGMRLTATYRYILSCHCEECNS

SEQ ID NO: 19 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSELKALRLRCSGGMRLTATYRYILSCHCEECNS

SEQ ID NO: 20 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLEALRLRCSGGMRLTATYRYILSCHCEECNS

SEQ ID NO: 21 - protein, 133 aa, Artificial sequence
  Synthetic
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMELTATYRYILSCHCEECNS